(19) United States
(12) Patent Application Publication
(10) Pub. No.: US 2026/0062378 A1
(43) Pub. Date: Mar. 5, 2026

(54) O-TERT-BUTYL-L-SERYL-O-TERT-BUTYL-L-SERYL-GLYCINE AND PREPARATION METHOD THEREOF

(71) Applicant: SICHUAN SHIFANG SANGAO BIOCHEMICAL INDUSTRIAL CO., LTD., Chengdu (CN)

(72) Inventors: Xiaolong FANG, Chengdu (CN); Hongguo LIANG, Chengdu (CN); Jianlong DENG, Chengdu (CN); Kaitai HE, Chengdu (CN); Jian YANG, Chengdu (CN); Mei LIU, Chengdu (CN)

(73) Assignee: SICHUAN SHIFANG SANGAO BIOCHEMICAL INDUSTRIAL CO., LTD., Chengdu (CN)

(21) Appl. No.: 19/313,770

(22) Filed: Aug. 28, 2025

(30) Foreign Application Priority Data
Aug. 29, 2024 (CN) 202411203400.2

(51) Int. Cl. C07C 237/12; C07C 231/14

(52) U.S. Cl. CPC C07C 237/12; C07C 231/14

(57) ABSTRACT

The present application provides O-tert-butyl-L-seryl-O-tert-butyl-L-seryl-glycine and a preparation method thereof. In the preparation process, N-benzyloxycarbonyl-O-tert-butyl-L-serine and O-tert-butyl-L-serine methyl ester are subjected to a condensation reaction to obtain an intermediate compound represented by formula (I), the compound represented by formula (I) and an alkaline compound are subjected to a saponification reaction to obtain an intermediate compound represented by formula (II), then the intermediate compound represented by formula (II) and glycine benzyl ester p-toluenesulfonate are subjected to a condensation reaction to obtain an intermediate compound represented by formula (III), and finally, the intermediate compound represented by formula (III) and hydrogen are subjected to a hydrogenation reaction to obtain the final product. The preparation method provided by the present application simplifies the process, features simple operation and mild reaction conditions, and reduces the difficulty of industrial production, and the final product has a high purity and a high yield.

### CROSS REFERENCE TO RELATED APPLICATIONS

This application claims the priority of Chinese Patent Application No. CN202411203400.2, filed with the China National Intellectual Property Administration on Aug. 29, 2024, and titled with “O-TERT-BUTYL-L-SERYL-O-TERT-BUTYL-L-SERYL-GLYCINE AND PREPARATION METHOD THEREOF”, which is hereby incorporated by reference.

### FIELD

The present application relates to the field of synthesis of pharmaceuticals, in particular to an O-tert-butyl-L-seryl-O-tert-butyl-L-seryl-glycine and preparation method thereof.

### BACKGROUND

Tirzepatide is a novel dual receptor agonist of GIP (gastric inhibitory polypeptide) and GLP-1 (glucagon-like peptide-1), which is used for the targeted treatment of diabetes, non-alcoholic steatohepatitis (NASH) and chronic weight management, with a half-life of 116.7 hours. Tirzepatide was developed by Eli Lilly and Company, and the marketing application for tirzepatide injection in China was accepted by the NMPA on Sep. 7, 2022, for improving blood glucose control in adults with type 2 diabetes. On Nov. 8, 2023, the U.S. FDA approved tirzepatide of Eli Lilly and Company for marketing, making it the first dual-target agonist (GIP/GLP-1) obesity treatment drug among similar marketed products. Therefore, tirzepatide has a good development prospect. O-tert-butyl-L-seryl-O-tert-butyl-L-seryl-glycine is an important intermediate in the synthesis of tirzepatide. Tirzepatide is finally obtained through multiple steps of reactions.

At present, there are no literature or patent reports on the preparation method of O-tert-butyl-L-seryl-O-tert-butyl-L-seryl-glycine. Although the applicant obtained the final compound O-tert-butyl-L-seryl-O-tert-butyl-L-seryl-glycine by adopting multi-step synthesis procedures during the research process, it contained many impurities, was not easy to purify, and had a low molar yield.

In view of this, there is an urgent need to develop an industrial synthetic route with simple process, mild reaction conditions and low cost for the preparation of O-tert-butyl-L-seryl-O-tert-butyl-L-seryl-glycine.

### SUMMARY

The problem solved by the present application is to provide a method for preparing O-tert-butyl-L-seryl-O-tert-butyl-L-seryl-glycine. This method simplifies the process and features mild reaction conditions. Furthermore, the O-tert-butyl-L-seryl-O-tert-butyl-L-seryl-glycine prepared by the present application has a high purity and a high yield.

In view of this, the present application provides O-tert-butyl-L-seryl-O-tert-butyl-L-seryl-glycine represented by formula (I)

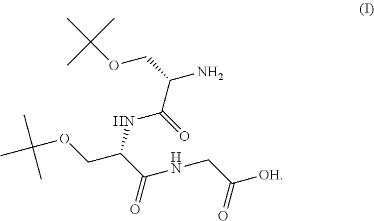 [SMILES: CC(C)(C)OC[C@H](NC(=O)[C@@H](N)COC(C)(C)C)C(=O)NCC(=O)O]

The present application further provides a method for preparing O-tert-butyl-L-seryl-O-tert-butyl-L-seryl-glycine, comprising the following steps:
S1) carrying out a condensation reaction on N-benzyloxycarbonyl-O-tert-butyl-L-serine and O-tert-butyl-L-serine methyl ester under the action of a condensing agent and an organic base to obtain N-benzyloxycarbonyl-O-tert-butyl-L-seryl-O-tert-butyl-L-serine methyl ester represented by formula (II);
S2) carrying out a saponification reaction on the N-benzyloxycarbonyl-O-tert-butyl-L-seryl-O-tert-butyl-L-serine methyl ester and an alkaline compound to obtain N-benzyloxycarbonyl-O-tert-butyl-L-seryl-O-tert-butyl-L-serine represented by formula (III);
S3) carrying out a condensation reaction on the N-benzyloxycarbonyl-O-tert-butyl-L-seryl-O-tert-butyl-L-serine and glycine benzyl ester p-toluenesulfonate under the action of a condensing agent and an organic base to obtain N-benzyloxycarbonyl-O-tert-butyl-L-seryl-O-tert-butyl-L-seryl-glycine benzyl ester represented by formula (IV);
S4) carrying out a hydrogenation reaction on the N-benzyloxycarbonyl-O-tert-butyl-L-seryl-O-tert-butyl-L-seryl-glycine benzyl ester and hydrogen under the action of a catalyst to obtain O-tert-butyl-L-seryl-O-tert-butyl-L-seryl-glycine represented by formula (I);

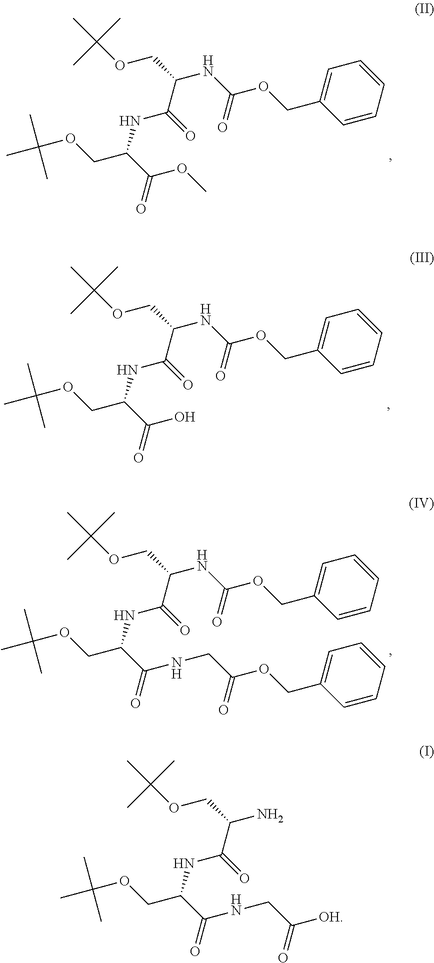 [SMILES: COC(=O)[C@H](COC(C)(C)C)NC(=O)[C@H](COC(C)(C)C)NC(=O)OCc1ccccc1 ; CC(C)(C)OC[C@H](NC(=O)[C@H](COC(C)(C)C)NC(=O)OCc1ccccc1)C(=O)O ; CC(C)(C)OC[C@H](NC(=O)OCc1ccccc1)C(=O)N[C@@H](COC(C)(C)C)C(=O)NCC(=O)OCc1ccccc1 ; CC(C)(C)OC[C@H](NC(=O)[C@@H](N)COC(C)(C)C)C(=O)NCC(=O)O]

In one embodiment, step S1) specifically is
mixing the N-benzyloxycarbonyl-O-tert-butyl-L-serine, the O-tert-butyl-L-serine methyl ester and the organic base with an organic solvent, then adding the condensing agent to carry out the condensation reaction to obtain the N-benzyloxycarbonyl-O-tert-butyl-L-seryl-O-tert-butyl-L-serine methyl ester.

In one embodiment, in step S1), a molar ratio of the N-benzyloxycarbonyl-O-tert-butyl-L-serine, the O-tert-butyl-L-serine methyl ester, the condensing agent, and the organic base is 1:(1-2):(1-1.5):(1-1.5).

In one embodiment, the condensing agent is selected from the group consisting of EDCl/HOBT, EDCl/HOSU, DCC/HOSU, HATU, HBTU and CDI, and the organic base is selected from the group consisting of triethylamine and N, N-diisopropylethylamine.

In one embodiment, the condensing agent is added at a temperature of 0-5° C., and the condensation reaction is carried out at a temperature of 0-50° C. for 12-24 h.

In one embodiment, the organic solvent is selected from the group consisting of ethyl acetate, dichloromethane, acetonitrile and tetrahydrofuran.

In one embodiment, step S2) specifically is:
mixing the N-benzyloxycarbonyl-O-tert-butyl-L-seryl-O-tert-butyl-L-serine methyl ester with an organic solvent, then adding a sodium hydroxide solution to carry out the saponification reaction to obtain the N-benzyloxycarbonyl-O-tert-butyl-L-seryl-O-tert-butyl-L-serine.

In one embodiment, in step S2), a molar ratio of the N-benzyloxycarbonyl-O-tert-butyl-L-seryl-O-tert-butyl-L-serine methyl ester to the sodium hydroxide is 1:(1-2).

In one embodiment, in step S2), the sodium hydroxide solution is added at a temperature of 0-5° C., and the saponification reaction is carried out at a temperature of 0-40° C. for 1-6 h.

In one embodiment, in step S2), a concentration of the sodium hydroxide solution is 5-60%.

In one embodiment, in step S2), the organic solvent is selected from the group consisting of acetonitrile, tetrahydrofuran, methanol and ethanol.

In one embodiment, step S3) specifically is:
mixing the N-benzyloxycarbonyl-O-tert-butyl-L-seryl-O-tert-butyl-L-serine, the glycine benzyl ester p-toluenesulfonate and the organic base with an organic solvent, then adding the condensing agent to carry out the condensation reaction to obtain the N-benzyloxycarbonyl-O-tert-butyl-L-seryl-O-tert-butyl-L-seryl-glycine benzyl ester.

In one embodiment, in step S3), a molar ratio of the N-benzyloxycarbonyl-O-tert-butyl-L-seryl-O-tert-butyl-L-serine, the glycine benzyl ester p-toluenesulfonate, the organic base, and the condensing agent is 1:(1-2):(1-1.5):(1-1.5).

In one embodiment, in step S3), the condensing agent is selected from the group consisting of EDCl/HOBT, EDCl/HOSU, DCC/HOSU, HATU, HBTU and CDI, and the organic base is selected from the group consisting of trimethylamine and N, N-diisopropylethylamine.

In one embodiment, in step S3), the condensing agent is added at a temperature of 0-5° C., and the condensation reaction is carried out at a temperature of 0-50° C. for 12-24 h.

In one embodiment, in step S3), the organic solvent is selected from the group consisting of ethyl acetate, dichloromethane, acetonitrile and tetrahydrofuran.

In one embodiment, step S4) specifically is:
mixing the N-benzyloxycarbonyl-O-tert-butyl-L-seryl-O-tert-butyl-L-seryl-glycine benzyl ester with an organic solvent, adding the catalyst, and then introducing hydrogen to carry out the hydrogenation reaction to obtain the O-tert-butyl-L-seryl-O-tert-butyl-L-seryl-glycine.

In one embodiment, in step S4), a mass ratio of the N-benzyloxycarbonyl-O-tert-butyl-L-seryl-O-tert-butyl-L-seryl-glycine benzyl ester to the catalyst is 1:(0.01-0.2).

Preferably, in step S4), the hydrogenation reaction is carried out at a temperature of 20-30° C. for 20-24 h.

In one embodiment, the organic solvent is selected from the group consisting of acetonitrile, tetrahydrofuran/water, methanol, ethanol, acetone/water and ethyl acetate/water, and the catalyst is selected from the group consisting of Pd/C, Raney nickel and Pd/BaSO₄.

The present application provides a method for preparing O-tert-butyl-L-seryl-O-tert-butyl-L-seryl-glycine, comprising first carrying out a condensation reaction on N-benzyloxycarbonyl-O-tert-butyl-L-serine and O-tert-butyl-L-serine methyl ester to obtain N-benzyloxycarbonyl-O-tert-butyl-L-seryl-O-tert-butyl-L-serine methyl ester, then carrying out a saponification reaction on the N-benzyloxycarbonyl-O-tert-butyl-L-seryl-O-tert-butyl-L-serine methyl ester with an alkaline compound, carrying out a condensation reaction on the resulting product with glycine benzyl ester p-toluenesulfonate, and finally carrying out a hydrogenation reaction on the resulting product with hydrogen to obtain O-tert-butyl-L-seryl-O-tert-butyl-L-seryl-glycine. In the present application, in the above process of preparing O-tert-butyl-L-seryl-O-tert-butyl-L-seryl-glycine, N-benzyloxycarbonyl-O-tert-butyl-L-serine is used as a raw material, and the tirzepatide intermediate O-tert-butyl-L-seryl-O-tert-butyl-L-seryl-glycine is synthesized through four steps, namely, sequential condensation reaction, saponification reaction, condensation reaction, and hydrogenation reaction, which simplifies the process, features simple operation and mild reaction conditions. Furthermore, the present application improves the yield and purity of O-tert-butyl-L-seryl-O-tert-butyl-L-seryl-glycine by regulating the specific process conditions in the above preparation steps.

### BRIEF DESCRIPTION OF DRAWINGS

FIG. 1 is an HPLC chromatogram of O-tert-butyl-L-seryl-O-tert-butyl-L-seryl-glycine prepared in Example 2 of the present application.

FIG. 2 is a hydrogen nuclear magnetic resonance spectrum of O-tert-butyl-L-seryl-O-tert-butyl-L-seryl-glycine prepared in Example 2 of the present application in CD₃OD.

FIG. 3 is a hydrogen nuclear magnetic resonance spectrum of O-tert-butyl-L-seryl-O-tert-butyl-L-seryl-glycine prepared in Example 2 of the present application in D₂O.

FIG. 4 is a hydrogen nuclear magnetic resonance spectrum of O-tert-butyl-L-seryl-O-tert-butyl-L-seryl-glycine prepared in Example 2 of the present application in DMSO.

### DETAILED DESCRIPTION

To further understand the present application, the following describes the embodiments of the present application in conjunction with examples. However, it should be understood that these descriptions are only for further illustrating the features and advantages of the present application, and not for limiting the claims of the present application.

Considering the demand in the prior art for an industrial synthetic route with a simple preparation process, mild reaction conditions, and low cost for the synthesis of tirzepatide intermediates, the present application provides O-tert-butyl-L-seryl-O-tert-butyl-L-seryl-glycine and preparation method thereof. In the preparation process, N-benzyloxycarbonyl-O-tert-butyl-L-serine, as a raw material, is reacted with other reagents through four steps of a condensation reaction, a saponification reaction, a condensation reaction, and a hydrogenation reaction to synthesize the tirzepatide intermediate O-tert-butyl-L-seryl-O-tert-butyl-L-seryl-glycine. This preparation method simplifies the process route, features simple operation and mild reaction conditions, and reduces the difficulty of industrial production. In the preparation process, by controlling the process conditions during preparation, the yield and purity of the tirzepatide intermediate O-tert-butyl-L-seryl-O-tert-butyl-L-seryl-glycine are further improved.

First, the present application provides O-tert-butyl-L-seryl-O-tert-butyl-L-seryl-glycine represented by formula (I)

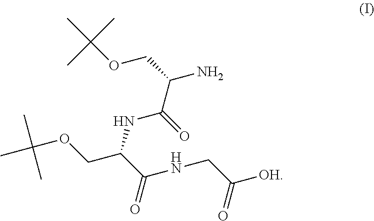 [SMILES: CC(C)(C)OC[C@H](NC(=O)[C@@H](N)COC(C)(C)C)C(=O)NCC(=O)O]

Further, in an embodiment, the present application provides a method for preparing O-tert-butyl-L-seryl-O-tert-butyl-L-seryl-glycine, comprising the following steps:
S1) carrying out a condensation reaction on N-benzyloxycarbonyl-O-tert-butyl-L-serine and O-tert-butyl-L-serine methyl ester under the action of a condensing agent and an organic base to obtain N-benzyloxycarbonyl-O-tert-butyl-L-seryl-O-tert-butyl-L-serine methyl ester represented by formula (II);
S2) carrying out a saponification reaction on the N-benzyloxycarbonyl-O-tert-butyl-L-seryl-O-tert-butyl-L-serine methyl ester and an alkaline compound to obtain N-benzyloxycarbonyl-O-tert-butyl-L-seryl-O-tert-butyl-L-serine represented by formula (III);
S3) carrying out a condensation reaction on the N-benzyloxycarbonyl-O-tert-butyl-L-seryl-O-tert-butyl-L-serine and glycine benzyl ester p-toluenesulfonate under the action of a condensing agent and an organic base to obtain N-benzyloxycarbonyl-O-tert-butyl-L-seryl-O-tert-butyl-L-seryl-glycine benzyl ester represented by formula (IV);
S4) carrying out a hydrogenation reaction on the N-benzyloxycarbonyl-O-tert-butyl-L-seryl-O-tert-butyl-L-seryl-glycine benzyl ester and hydrogen under the action of a catalyst to obtain O-tert-butyl-L-seryl-O-tert-butyl-L-seryl-glycine represented by formula (I);

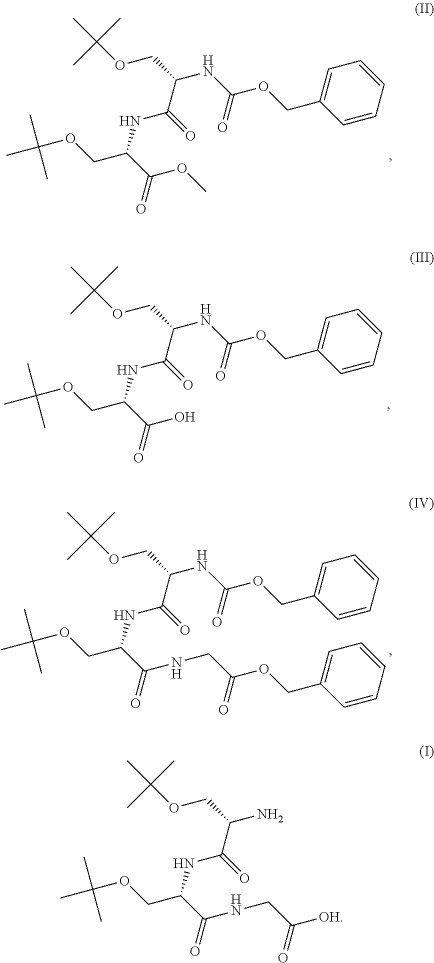 [SMILES: COC(=O)[C@H](COC(C)(C)C)NC(=O)[C@H](COC(C)(C)C)NC(=O)OCc1ccccc1 ; CC(C)(C)OC[C@H](NC(=O)[C@H](COC(C)(C)C)NC(=O)OCc1ccccc1)C(=O)O ; CC(C)(C)OC[C@H](NC(=O)OCc1ccccc1)C(=O)N[C@@H](COC(C)(C)C)C(=O)NCC(=O)OCc1ccccc1 ; CC(C)(C)OC[C@H](NC(=O)[C@@H](N)COC(C)(C)C)C(=O)NCC(=O)O]

In the present application, in the preparation process of the tirzepatide intermediate O-tert-butyl-L-seryl-O-tert-butyl-L-seryl-glycine, N-benzyloxycarbonyl-O-tert-butyl-L-serine and O-tert-butyl-L-serine methyl ester are used as raw materials to carry out the condensation reaction under the conditions of the condensing agent and the organic base, thereby obtaining the N-benzyloxycarbonyl-O-tert-butyl-L-seryl-O-tert-butyl-L-serine methyl ester.

The process specifically comprises the following steps: mixing the N-benzyloxycarbonyl-O-tert-butyl-L-serine, the O-tert-butyl-L-serine methyl ester and the organic base with an organic solvent uniformly, then adding the condensing agent and stirring to carry out the condensation reaction, and carrying out post-treatment to obtain the intermediate compound N-benzyloxycarbonyl-O-tert-butyl-L-seryl-O-tert-butyl-L-serine methyl ester as an oily substance.

The reaction formula of the above process is specifically as follows:

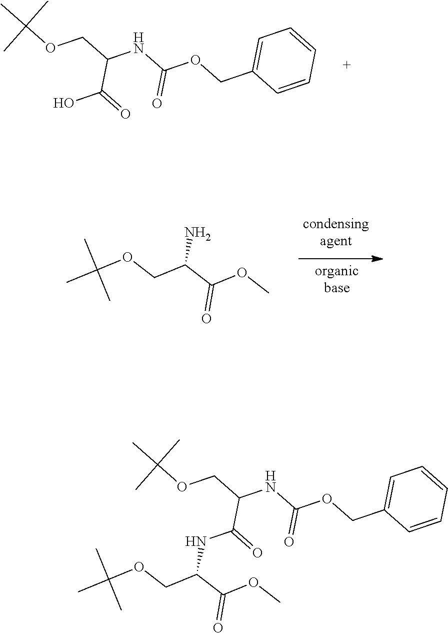 [SMILES: CC(C)(C)OCC(NC(=O)OCc1ccccc1)C(=O)O ; COC(=O)[C@@H](N)COC(C)(C)C ; COC(=O)[C@H](COC(C)(C)C)NC(=O)C(COC(C)(C)C)NC(=O)OCc1ccccc1]

In the above process, a molar ratio of the N-benzyloxycarbonyl-O-tert-butyl-L-serine, the O-tert-butyl-L-serine methyl ester, the condensing agent, and the organic base is 1:(1-2):(1-1.5):(1-1.5). Specifically, the molar ratio of the N-benzyloxycarbonyl-O-tert-butyl-L-serine, the O-tert-butyl-L-serine methyl ester, the condensing agent, and the organic base is 1:(1.2-1.8):(1.1-1.4):(1.1-1.4). More specifically, the molar ratio of the N-benzyloxycarbonyl-O-tert-butyl-L-serine, the O-tert-butyl-L-serine methyl ester, the condensing agent, and the organic base is 1:1.5:1.2:1.4. The condensing agent is added at a temperature of 0-5° C. Specifically, the condensing agent is added at a temperature of 1-3° C. If the addition temperature of the condensing agent is too high, it will lead to an increase in impurities. The condensation reaction is carried out at a temperature of 0-50° C. Specifically, the condensation reaction is carried out at a temperature of 0-10° C., 10-20° C., 20-25° C., 25-30° C., 30-35° C., 35-40° C. or 40-50° C. In one embodiment, the condensation reaction is carried out at a temperature of 25-35° C. If the temperature of the condensation reaction is too low, the reaction is incomplete, resulting in low yield, and if the temperature of the condensation reaction is too high, the purity is reduced. The condensation reaction is carried out for 12-24 h. Specifically, the condensation reaction is carried out for 18-20 h. The organic base is selected from the group consisting of triethylamine and N, N-diisopropylethylamine. The condensing agent is selected from the group consisting of EDCl/HOBT, EDCl/HOSU, DCC/HOSU, HATU, HBTU and CDI. Further, the condensing agent is selected from the group consisting of EDCl/HOBT, EDCl/HOSU, HATU and HBTU. The condensing agent selected from the above reagents can make the yield and purity of the product higher. In the above condensing agents, EDCl/HOBT means that EDCl and HOBT are added simultaneously, and similarly, EDCl/HOSU also means that EDCl and HOSU are added simultaneously. The organic solvent is selected from the group consisting of ethyl acetate, dichloromethane, acetonitrile and tetrahydrofuran. Specifically, the organic solvent is selected from the group consisting of ethyl acetate and acetonitrile. Under the same conditions, the selection of the above organic solvent is beneficial to improve the yield and purity of the product.

In the present application, the above intermediate product N-benzyloxycarbonyl-O-tert-butyl-L-seryl-O-tert-butyl-L-serine methyl ester and the alkaline compound are subjected to the saponification reaction to obtain the N-benzyloxycarbonyl-O-tert-butyl-L-seryl-O-tert-butyl-L-serine.

The process specifically comprises the following steps: mixing the N-benzyloxycarbonyl-O-tert-butyl-L-seryl-O-tert-butyl-L-serine methyl ester with the organic solvent uniformly, then adding the sodium hydroxide solution and stirring to carry out the saponification reaction, and carrying out post-treatment to obtain the intermediate compound the N-benzyloxycarbonyl-O-tert-butyl-L-seryl-O-tert-butyl-L-serine as an oily substance.

The reaction formula of the above process is specifically as follows:

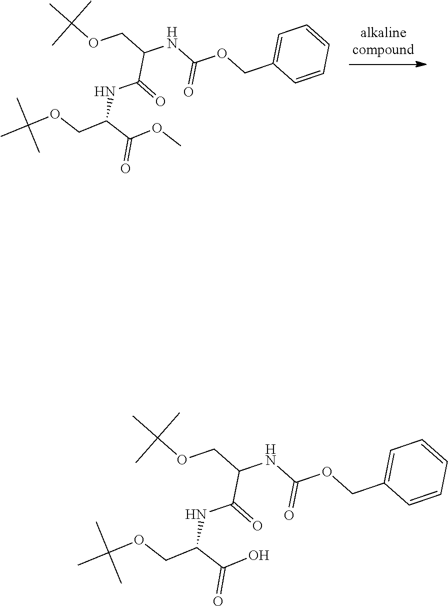 [SMILES: COC(=O)[C@H](COC(C)(C)C)NC(=O)C(COC(C)(C)C)NC(=O)OCc1ccccc1 ; CC(C)(C)OCC(NC(=O)OCc1ccccc1)C(=O)N[C@@H](COC(C)(C)C)C(=O)O]

In the above process, a molar ratio of the N-benzyloxycarbonyl-O-tert-butyl-L-seryl-O-tert-butyl-L-serine methyl ester to the sodium hydroxide is 1:(1-2). Specifically, the molar ratio of the N-benzyloxycarbonyl-O-tert-butyl-L-seryl-O-tert-butyl-L-serine methyl ester to the sodium hydroxide is 1:(1.2-1.8). More specifically, the molar ratio of the N-benzyloxycarbonyl-O-tert-butyl-L-seryl-O-tert-butyl-L-serine methyl ester to the sodium hydroxide is 1:1.5. The sodium hydroxide solution is added at a temperature of 0-5° C. Specifically, the sodium hydroxide solution is added at a temperature of 3-4° C. When the sodium hydroxide solution is added, the system temperature is increased, and if the adding temperature of the sodium hydroxide solution is not controlled, the reaction temperature is too high and impurities are increased. A concentration of the sodium hydroxide solution is 5-60%. Specifically, the concentration of the sodium hydroxide solution is 5-30%. More specifically, the concentration of the sodium hydroxide solution is 10%, 20% and 30%. If the concentration of the sodium hydroxide is too high, the purity of the product will be reduced. The saponification reaction is carried out at a temperature of 0-40° C. Specifically, the saponification reaction is carried out at a temperature of 0-10° C., 10-15° C., 15-20° C. or 20-25° C. Further, the saponification reaction is carried out at a temperature of 15-25° C. More specifically, the saponification reaction is carried out at a temperature of 15-20° C. or 20-25° C. If the temperature of the saponification reaction is low, the reaction time is long, impurities are increased. The above temperature can ensure quick reaction, excessive impurities are not increased, and the purity of the product is ensured. The saponification reaction is carried out for 1-6 h. Specifically, the saponification reaction is carried out for 2-4 h. More specifically, the saponification reaction is carried out for 3 h or 4 h. The organic solvent is selected from the group consisting of acetonitrile, tetrahydrofuran, methanol and ethanol.

According to the present application, the obtained product N-benzyloxycarbonyl-O-tert-butyl-L-seryl-O-tert-butyl-L-serine and glycine benzyl ester p-toluenesulfonate are subjected to the condensation reaction under the action of the condensing agent and the organic base to obtain the N-benzyloxycarbonyl-O-tert-butyl-L-seryl-O-tert-butyl-L-seryl-glycine benzyl ester.

The process specifically comprises the following steps: mixing the N-benzyloxycarbonyl-O-tert-butyl-L-seryl-O-tert-butyl-L-serine, the glycine benzyl ester p-toluenesulfonate and the organic base with the organic solvent, then adding the condensing agent and mixing to carry out the condensation reaction, and carrying out post-treatment to obtain the intermediate compound N-benzyloxycarbonyl-O-tert-butyl-L-seryl-O-tert-butyl-L-seryl-glycine benzyl ester as an oily substance.

The reaction formula of the above process is specifically as follows:

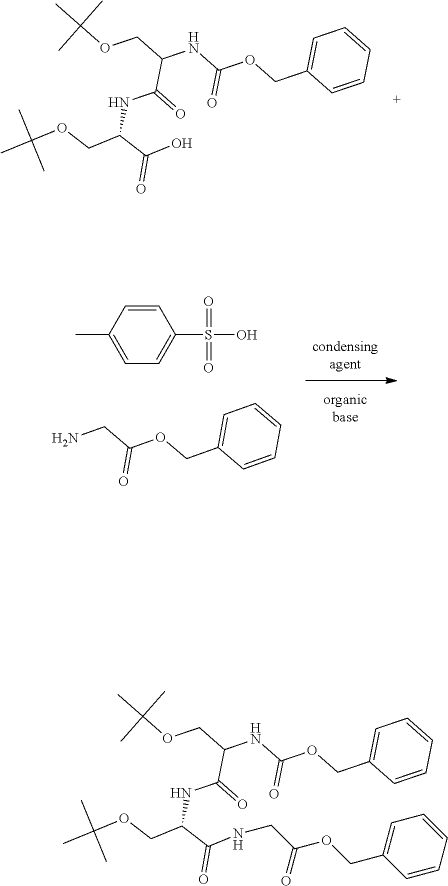 [SMILES: CC(C)(C)OCC(NC(=O)OCc1ccccc1)C(=O)N[C@@H](COC(C)(C)C)C(=O)O ; Cc1ccc(S(=O)(=O)O)cc1 ; NCC(=O)OCc1ccccc1 ; CC(C)(C)OCC(NC(=O)OCc1ccccc1)C(=O)N[C@@H](COC(C)(C)C)C(=O)NCC(=O)OCc1ccccc1]

In the above process, a molar ratio of the N-benzyloxycarbonyl-O-tert-butyl-L-seryl-O-tert-butyl-L-serine, the glycine benzyl ester p-toluenesulfonate, the condensing agent and the organic base is 1:(1-2):(1-1.5):(1-1.5).

Specifically, the molar ratio of the N-benzyloxycarbonyl-O-tert-butyl-L-seryl-O-tert-butyl-L-serine, the glycine benzyl ester p-toluenesulfonate, the condensing agent and the organic base is 1:(1.2-1.8):(1.1-1.4):(1.1-1.4). More specifically, the molar ratio of the N-benzyloxycarbonyl-O-tert-butyl-L-seryl-O-tert-butyl-L-serine, the glycine benzyl ester p-toluenesulfonate, the condensing agent and the organic base is 1:1.5:1.2:1.4. In the present application, the glycine benzyl ester p-toluenesulfonate is used as a key raw material. If glycine methyl ester hydrochloride or other materials are used as reaction raw materials, the reaction steps will be increased, and the final product will have more impurities, be difficult to purify and have lower molar yield. The condensing agent is added at a temperature controlled to be 0-5° C. Specifically, the condensing agent is added at a temperature controlled to be 2-4° C. If the addition temperature of the condensing agent is too high, it will lead to an increase in impurities. The condensation reaction is carried out at a temperature of 0-50° C. Specifically, the condensation reaction is carried out at a temperature of 0-10° C., 10-20° C., 20-25° C., 25-30° C., 30-35° C., 35-40° C. or 40-50° C. In one embodiment, the condensation reaction is carried out at a temperature of 25-35° C. Further, the condensation reaction is carried out at a temperature of 30-35° C. If the temperature of the condensation reaction is too low, the reaction is not complete, resulting in a low yield, and if the temperature of the condensation reaction is too high, resulting in a low purity. The condensation reaction is carried out for 12-24 h. Specifically, the condensation reaction is carried out for 18-20 h. The organic base is selected from the group consisting of trimethylamine and N, N-diisopropylethylamine. The condensing agent is selected from the group consisting of EDCl/HOBT, EDCl/HOSU, DCC/HOSU, HATU, HBTU and CDI. Further, the condensing agent is selected from the group consisting of EDCl/HOBT, EDCl/HOSU, HATU and HBTU. The condensing agent selected from the above reagents can make the yield and purity of the product higher. In the above condensing agents, EDCl/HOBT means that EDCl and HOBT are added simultaneously, and similarly, EDCl/HOSU also means that EDCl and HOSU are added simultaneously. The organic solvent is selected from the group consisting of ethyl acetate, dichloromethane, acetonitrile and tetrahydrofuran. Specifically, the organic solvent is selected from the group consisting of ethyl acetate and acetonitrile. Under the same conditions, the selection of the above organic solvent is beneficial to improve the yield and purity of the product. In the present application, the condensation reaction in this process and the condensation reaction in the step S1) may be performed under the same or different process conditions, which is not particularly limited in this application.

In the present application, finally, the obtained intermediate product N-benzyloxycarbonyl-O-tert-butyl-L-seryl-O-tert-butyl-L-seryl-glycine benzyl ester and hydrogen are subjected to the hydrogenation reaction under the action of the catalyst to obtain O-tert-butyl-L-seryl-O-tert-butyl-L-seryl-glycine.

The process specifically comprises the following steps: mixing the N-benzyloxycarbonyl-O-tert-butyl-L-seryl-O-tert-butyl-L-seryl-glycine benzyl ester with the organic solvent uniformly, adding the catalyst, introducing hydrogen under stirring to carry out the hydrogenation reaction, and carrying out post-treatment to obtain the target compound O-tert-butyl-L-seryl-O-tert-butyl-L-seryl-glycine. The reaction formula of the above process is specifically as follows:

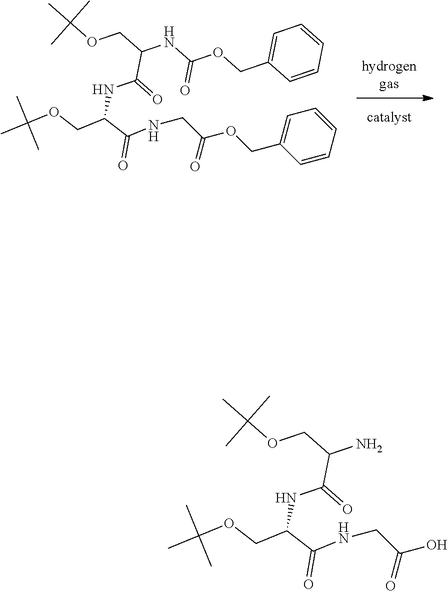 [SMILES: CC(C)(C)OCC(NC(=O)OCc1ccccc1)C(=O)N[C@@H](COC(C)(C)C)C(=O)NCC(=O)OCc1ccccc1 ; CC(C)(C)OCC(N)C(=O)N[C@@H](COC(C)(C)C)C(=O)NCC(=O)O]

In the above process, a mass ratio of the N-benzyloxycarbonyl-O-tert-butyl-L-seryl-O-tert-butyl-L-seryl-glycine benzyl ester to the catalyst is 1:(0.01-0.2). Specifically, the mass ratio of the N-benzyloxycarbonyl-O-tert-butyl-L-seryl-O-tert-butyl-L-seryl-glycine benzyl ester to the catalyst is 1:(0.07-0.15). More specifically, the mass ratio of the N-benzyloxycarbonyl-O-tert-butyl-L-seryl-O-tert-butyl-L-seryl-glycine benzyl ester to the catalyst is 1:0.1. The hydrogenation reaction is carried out at a temperature of 20-30° C. Specifically, the hydrogenation reaction is carried out at a temperature of 23-27° C. If the temperature of the hydrogenation reaction is too low, the reaction time is long, and if the reaction temperature is too high, impurities are increased, so that the impurities are not easy to remove. The hydrogenation reaction is carried out for 20-24 h. Specifically, the hydrogenation reaction is carried out for 21-23 h. The organic solvent is selected from the group consisting of acetonitrile, tetrahydrofuran/water, methanol, ethanol, acetone/water and ethyl acetate/water. Specifically, the organic solvent is methanol. The catalyst is selected from the group consisting of Pd/C, Raney nickel and Pd/BaSO₄. Specifically, the catalyst is Pd/C.

For further understanding of the present application, the following examples are given to illustrate the preparation of O-tert-butyl-L-seryl-O-tert-butyl-L-seryl-glycine according to the present application, and the scope of the present application is not limited by the following examples.

The raw materials used in the application are all industrial raw materials which can be purchased in the market.

### Example 1

### (1) Preparation of N-benzyloxycarbonyl-O-tert-butyl-L-seryl-O-tert-butyl-L-serine methyl ester

100 g of N-benzyloxycarbonyl-O-tert-butyl-L-serine was added into 800 g of ethyl acetate, and stirred to dissolve completely, and then 90 g of O-tert-butyl-L-serine methyl ester and 51.3 g of trimethylamine were added. After stirring uniformly, the mixture was cooled to 3° C., and then 9.1 g of HOBT and 78 g of EDCl were added. The mixture was stirred to react for 30 minutes, and heated to 33° C. to react for 18 hours. TLC showed that N-carbobenzoxy-O-tert-butyl-L-serine had completely reacted. 400 g of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 168 g of N-benzyloxycarbonyl-O-tert-butyl-L-seryl-O-tert-butyl-L-serine methyl ester as an oily substance.

### (2) Preparation of N-benzyloxycarbonyl-O-tert-butyl-L-seryl-O-tert-butyl-L-serine

168 g of the N-benzyloxycarbonyl-O-tert-butyl-L-seryl-O-tert-butyl-L-serine methyl ester as the oily substance was added into 150 g of tetrahydrofuran and 300 g of water. The mixture was cooled to 4° C., and 67.6 g of 30% sodium hydroxide aqueous solution was slowly added. After stirring uniformly, the mixture was heated to 23° C. to react for 3 hours. TLC showed that N-benzyloxycarbonyl-O-tert-butyl-L-seryl-O-tert-butyl-L-serine methyl ester had completely reacted. 400 g of ethyl acetate was added into the system. The pH value of the system was adjusted to 2-3 by using a 6N hydrochloric acid aqueous solution, and then liquids were separated. The ethyl acetate layer was collected. The ethyl acetate layer was washed with saturated sodium chloride for 3 times. After the washing was completed, the ethyl acetate layer was dried over sodium sulfate, filtered, and concentrated to dryness to obtain 152 g of N-benzyloxycarbonyl-O-tert-butyl-L-seryl-O-tert-butyl-L-serine as an oily substance.

### (3) Preparation of N-benzyloxycarbonyl-O-tert-butyl-L-seryl-O-tert-butyl-L-seryl-glycine benzyl ester

152 g of N-benzyloxycarbonyl-O-tert-butyl-L-seryl-O-tert-butyl-L-serine was added into 1200 g of ethyl acetate, and stirred to dissolve completely, and then 121 g of glycine benzyl ester p-toluenesulfonate and 48 g of triethylamine were added. After stirring uniformly, the mixture was cooled to 2° C., and then 9.3 g of HOBT and 79 g of EDCl were added. The mixture was stirred to react for 30 minutes, and heated to 33° C. to react for 18 hours. TLC showed that N-benzyloxycarbonyl-O-tert-butyl-L-seryl-O-tert-butyl-L-serine had completely reacted. 400 g of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 177 g of N-benzyloxycarbonyl-O-tert-butyl-L-seryl-O-tert-butyl-L-seryl-glycine benzyl ester as an oily substance.

### (4) Preparation of O-tert-butyl-L-seryl-O-tert-butyl-L-seryl-glycine

177 g of N-benzyloxycarbonyl-O-tert-butyl-L-seryl-O-tert-butyl-L-seryl-glycine benzyl ester was added to 1060 g of methanol, and stirred to dissolve completely, and then 17.7 g of Pd/C was added. The temperature was controlled at 23° C. The system was replaced with nitrogen, and then hydrogen was introduced. After reacting for 23 hours, TLC showed that N-benzyloxycarbonyl-O-tert-butyl-L-seryl-O-tert-butyl-L-seryl-glycine benzyl ester had completely reacted. After filtering out the Pd/C, the filtrate was concentrated until a large amount of solid precipitated. The system was cooled to 2° C. for crystallization for 3 hours. The filter cake was rinsed with a small amount of methanol, and dried by blowing at 50° C. to dryness to obtain 89 g of O-tert-butyl-L-seryl-O-tert-butyl-L-seryl-glycine. The results show that: molar yield: 73%; HPLC: 99.48%; isomers: not detected.

### Example 2

### Preparation of N-benzyloxycarbonyl-O-tert-butyl-L-seryl-O-tert-butyl-L-serine methyl ester

(1) 1.7 kg of N-benzyloxycarbonyl-O-tert-butyl-L-serine was added into 13.6 kg of ethyl acetate, and stirred to dissolve completely, and then 1.62 kg of O-tert-butyl-L-serine methyl ester and 0.87 kg of trimethylamine were added. After stirring uniformly, the mixture was cooled to 1° C., and then 0.155 kg of HOBT and 1.32 kg of EDCl were added. The mixture was stirred to react for 30 minutes, and heated to 33° C. to react for 18 hours. TLC showed that N-carbobenzoxy-O-tert-butyl-L-serine had completely reacted. 7 kg of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 2.4 kg of N-benzyloxycarbonyl-O-tert-butyl-L-seryl-O-tert-butyl-L-serine methyl ester as an oily substance.

### (2) Preparation of N-benzyloxycarbonyl-O-tert-butyl-L-seryl-O-tert-butyl-L-serine

2.4 kg of the N-benzyloxycarbonyl-O-tert-butyl-L-seryl-O-tert-butyl-L-serine methyl ester as the oily substance was added into 4.8 kg of tetrahydrofuran and 12 kg of water. The mixture was cooled to 3° C., and 1 kg of 30% sodium hydroxide aqueous solution was slowly added. After stirring uniformly, the mixture was heated to 22° C. to react for 3 hours. TLC showed that N-benzyloxycarbonyl-O-tert-butyl-L-seryl-O-tert-butyl-L-serine methyl ester had completely reacted. 6 kg of ethyl acetate was added into the system. The pH value of the system was adjusted to 2-3 by using a 6N hydrochloric acid aqueous solution, and then liquids were separated. The ethyl acetate layer was collected. The ethyl acetate layer was washed with saturated sodium chloride for 3 times. After the washing was completed, the ethyl acetate layer was dried over sodium sulfate, filtered, and concentrated to dryness to obtain 2.2 kg of N-benzyloxycarbonyl-O-tert-butyl-L-seryl-O-tert-butyl-L-serine as an oily substance.

### (3) Preparation of N-benzyloxycarbonyl-O-tert-butyl-L-seryl-O-tert-butyl-L-seryl-glycine benzyl ester

2.2 kg of N-benzyloxycarbonyl-O-tert-butyl-L-seryl-O-tert-butyl-L-serine was added into 17.4 kg of ethyl acetate, and stirred to dissolve completely, and then 1.927 kg of glycine benzyl ester p-toluenesulfonate and 0.72 kg of triethylamine were added. After stirring uniformly, the mixture was cooled to 4° C., and then 0.134 kg of HOBT and 1.043 kg of EDCl were added. The mixture was stirred to react for 30 minutes, and heated to 33° C. to react for 18 hours. TLC showed that N-benzyloxycarbonyl-O-tert-butyl-L-seryl-O-tert-butyl-L-serine had completely reacted. 10 kg of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 2.8 kg of N-benzyloxycarbonyl-O-tert-butyl-L-seryl-O-tert-butyl-L-seryl-glycine benzyl ester as an oily substance.

### (4) Preparation of O-tert-butyl-L-seryl-O-tert-butyl-L-seryl-glycine

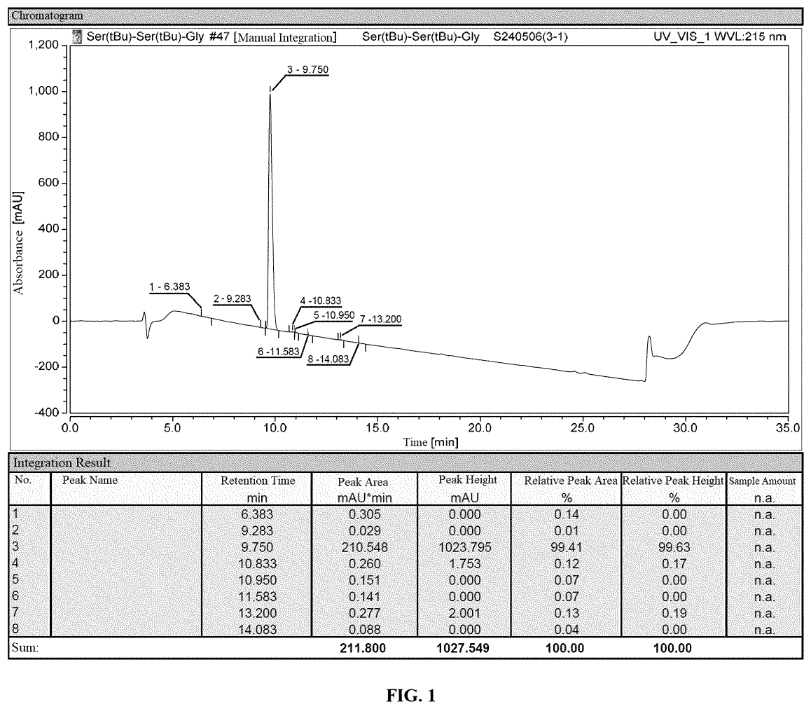

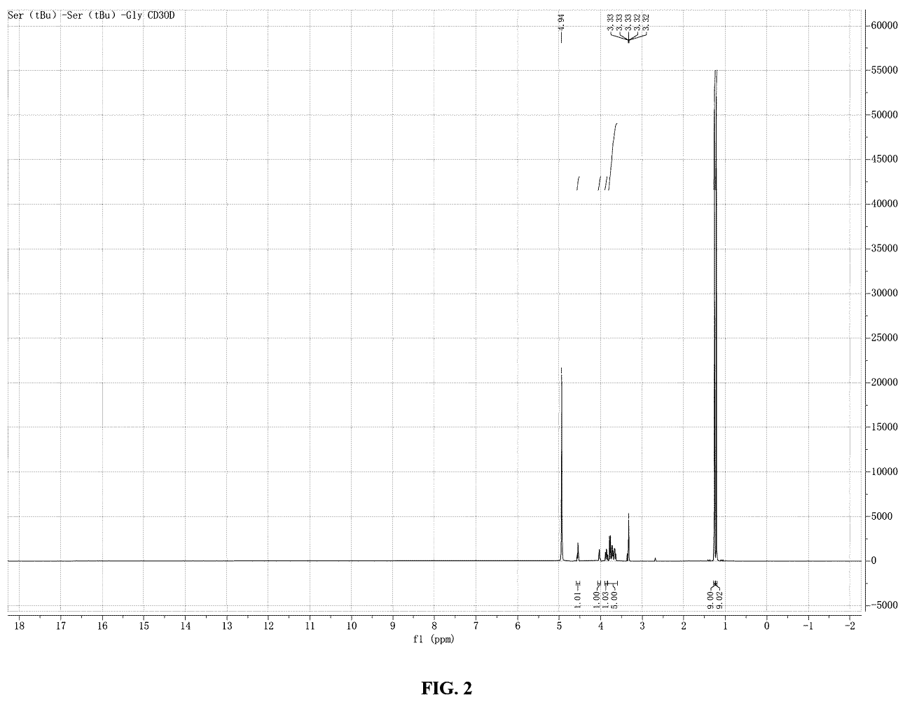

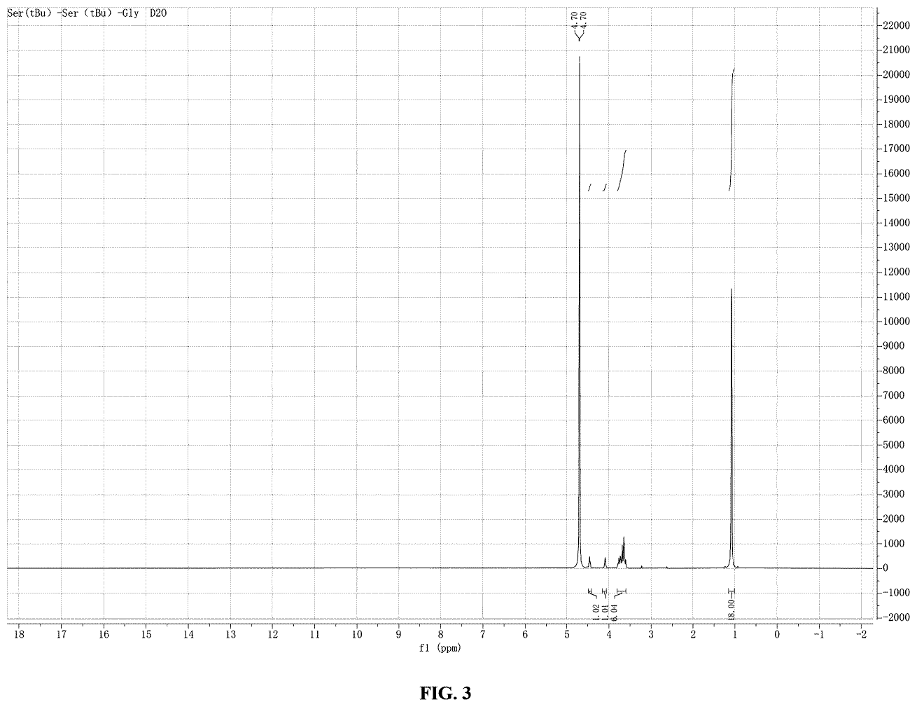

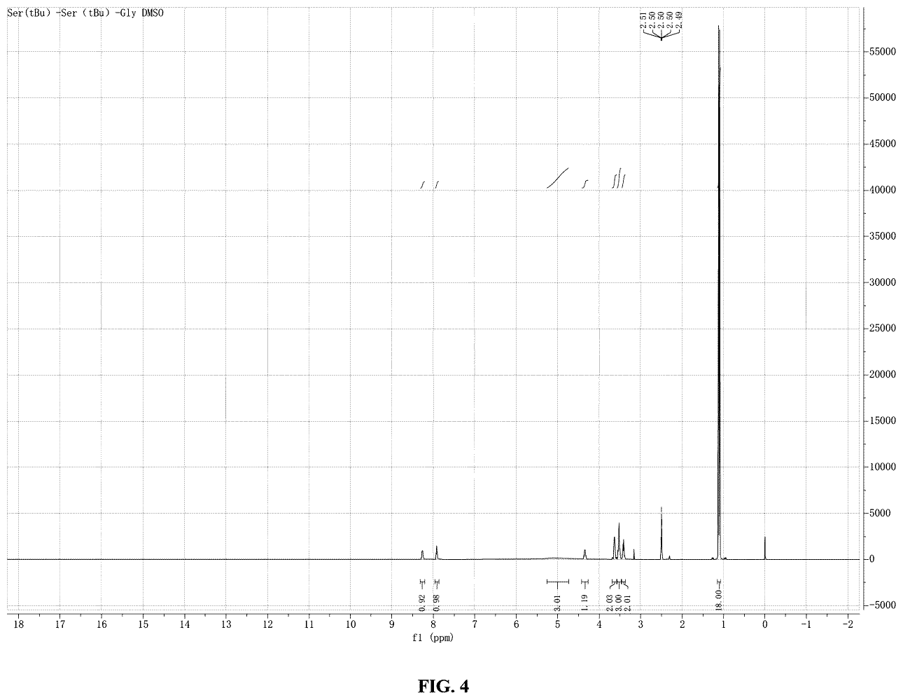

2.8 kg of N-benzyloxycarbonyl-O-tert-butyl-L-seryl-O-tert-butyl-L-seryl-glycine benzyl ester was added to 12 kg of methanol, and stirred to dissolve completely, and then 0.196 kg of Pd/C was added. The temperature was controlled at 23° C. The system was replaced with nitrogen, and then hydrogen was introduced. After reacting for 23 hours, TLC showed that N-benzyloxycarbonyl-O-tert-butyl-L-seryl-O-tert-butyl-L-seryl-glycine benzyl ester had completely reacted. After filtering out the Pd/C, the filtrate was concentrated until a large amount of solid precipitated. The system was cooled to 4° C. for crystallization for 3 hours. The filter cake was rinsed with a small amount of methanol, and dried by blowing at 50° C. to dryness to obtain 1.477 kg of O-tert-butyl-L-seryl-O-tert-butyl-L-seryl-glycine. The results show that: molar yield: 71%; HPLC: 99.41% (as shown in FIG. 1); isomers: not detected. The nuclear magnetic resonance spectra are shown in FIGS. 2, 3, and 4. FIGS. 2, 3, and 4 are the proton spectra of the final product in different solvents, respectively.

### Example 3

### (1) Preparation of N-benzyloxycarbonyl-O-tert-butyl-L-seryl-O-tert-butyl-L-serine methyl ester

100 g of N-benzyloxycarbonyl-O-tert-butyl-L-serine was added into 800 g of ethyl acetate, and stirred to dissolve completely, and then 90 g of O-tert-butyl-L-serine methyl ester and 61.2 g of N, N-diisopropylethylamine were added. After stirring uniformly, the mixture was cooled to 3° C., and then 9.1 g of HOBT and 78 g of EDCl were added. The mixture was stirred to react for 30 minutes, and heated to 33° C. to react for 18 hours. TLC showed that N-carbobenzoxy-O-tert-butyl-L-serine had completely reacted. 400 g of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 164 g of N-benzyloxycarbonyl-O-tert-butyl-L-seryl-O-tert-butyl-L-serine methyl ester as an oily substance.

### (2) Preparation of N-benzyloxycarbonyl-O-tert-butyl-L-seryl-O-tert-butyl-L-serine

164 g of the N-benzyloxycarbonyl-O-tert-butyl-L-seryl-O-tert-butyl-L-serine methyl ester as the oily substance was added into 160 g of tetrahydrofuran and 300 g of water. The mixture was cooled to 4° C., and 72.4 g of 30% sodium hydroxide aqueous solution was slowly added. After stirring uniformly, the mixture was heated to 23° C. to react for 3 hours. TLC showed that N-benzyloxycarbonyl-O-tert-butyl-L-seryl-O-tert-butyl-L-serine methyl ester had completely reacted. 400 g of ethyl acetate was added into the system. The pH value of the system was adjusted to 2-3 by using a 6N hydrochloric acid aqueous solution, and then liquids were separated. The ethyl acetate layer was collected. The ethyl acetate layer was washed with saturated sodium chloride for 3 times. After the washing was completed, the ethyl acetate layer was dried over sodium sulfate, filtered, and concentrated to dryness to obtain 150 g of N-benzyloxycarbonyl-O-tert-butyl-L-seryl-O-tert-butyl-L-serine as an oily substance.

### (3) Preparation of N-benzyloxycarbonyl-O-tert-butyl-L-seryl-O-tert-butyl-L-seryl-glycine benzyl ester

150 g of N-benzyloxycarbonyl-O-tert-butyl-L-seryl-O-tert-butyl-L-serine was added into 1200 g of ethyl acetate, and stirred to dissolve completely, and then 133 g of glycine benzyl ester p-toluenesulfonate and 61.8 g of N, N-diisopropylethylamine were added. After stirring uniformly, the mixture was cooled to 2° C., and then 9.3 g of HOBT and 79 g of EDCl were added. The mixture was stirred to react for 30 minutes, and heated to 33° C. to react for 18 hours. TLC showed that N-benzyloxycarbonyl-O-tert-butyl-L-seryl-O-tert-butyl-L-serine had completely reacted. 400 g of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 178 g of N-benzyloxycarbonyl-O-tert-butyl-L-seryl-O-tert-butyl-L-seryl-glycine benzyl ester as an oily substance.

### (4) Preparation of O-tert-butyl-L-seryl-O-tert-butyl-L-seryl-glycine

178 g of N-benzyloxycarbonyl-O-tert-butyl-L-seryl-O-tert-butyl-L-seryl-glycine benzyl ester was added to 1060 g of methanol, and stirred to dissolve completely, and then 17.8 g of Pd/C was added. The temperature was controlled at 23° C. The system was replaced with nitrogen, and then hydrogen was introduced. After reacting for 23 hours, TLC showed that N-benzyloxycarbonyl-O-tert-butyl-L-seryl-O-tert-butyl-L-seryl-glycine benzyl ester had completely reacted. After filtering out the Pd/C, the filtrate was concentrated until a large amount of solid precipitated. The system was cooled to 2° C. for crystallization for 3 hours. The filter cake was rinsed with a small amount of methanol, and dried by blowing at 50° C. to dryness to obtain 86 g of O-tert-butyl-L-seryl-O-tert-butyl-L-seryl-glycine. The results show that: molar yield: 70%; HPLC: 99.43%; isomers: not detected.

### Example 4

### (1) Preparation of N-benzyloxycarbonyl-O-tert-butyl-L-seryl-O-tert-butyl-L-serine methyl ester

100 g of N-benzyloxycarbonyl-O-tert-butyl-L-serine was added into 800 g of ethyl acetate, and stirred to dissolve completely, and then 90 g of O-tert-butyl-L-serine methyl ester and 51.3 g of trimethylamine were added. After stirring uniformly, the mixture was cooled to 3° C., and then 9.1 g of HOBT and 78 g of EDCl were added. The mixture was reacted at 0-5° C. for 18 hours. TLC showed that N-carbobenzoxy-O-tert-butyl-L-serine had not completely reacted. 400 g of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 79 g of N-benzyloxycarbonyl-O-tert-butyl-L-seryl-O-tert-butyl-L-serine methyl ester as an oily substance.

### (2) Preparation of N-benzyloxycarbonyl-O-tert-butyl-L-seryl-O-tert-butyl-L-serine

79 g of the N-benzyloxycarbonyl-O-tert-butyl-L-seryl-O-tert-butyl-L-serine methyl ester as the oily substance was added into 80 g of tetrahydrofuran and 150 g of water. The mixture was cooled to 4° C., and 31 g of 30% sodium hydroxide aqueous solution was slowly added. After stirring uniformly, the mixture was reacted at 0-5° C. for 23 h. TLC showed that N-benzyloxycarbonyl-O-tert-butyl-L-seryl-O-tert-butyl-L-serine methyl ester still had not completely reacted. 400 g of ethyl acetate was added into the system. The pH value of the system was adjusted to 2-3 by using a 6N hydrochloric acid aqueous solution, and then liquids were separated. The ethyl acetate layer was collected. The ethyl acetate layer was washed with saturated sodium chloride for 3 times. After the washing was completed, the ethyl acetate layer was dried over sodium sulfate, filtered, and concentrated to dryness to obtain 71 g of N-benzyloxycarbonyl-O-tert-butyl-L-seryl-O-tert-butyl-L-serine as an oily substance.

### (3) Preparation of N-benzyloxycarbonyl-O-tert-butyl-L-seryl-O-tert-butyl-L-seryl-glycine benzyl ester

71 g of N-benzyloxycarbonyl-O-tert-butyl-L-seryl-O-tert-butyl-L-serine was added into 560 g of ethyl acetate, and stirred to dissolve completely, and then 56 g of glycine benzyl ester p-toluenesulfonate and 22 g of triethylamine were added. After stirring uniformly, the mixture was cooled to 2° C., and then 4.3 g of HOBT and 37 g of EDCl were added. The mixture was reacted at 0-5° C. for 18 hours. TLC showed that N-benzyloxycarbonyl-O-tert-butyl-L-seryl-O-tert-butyl-L-serine had not completely reacted. 200 g of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 41 g of N-benzyloxycarbonyl-O-tert-butyl-L-seryl-O-tert-butyl-L-seryl-glycine benzyl ester as an oily substance.

### (4) Preparation of O-tert-butyl-L-seryl-O-tert-butyl-L-seryl-glycine

41 g of N-benzyloxycarbonyl-O-tert-butyl-L-seryl-O-tert-butyl-L-seryl-glycine benzyl ester was added to 250 g of methanol, and stirred to dissolve completely, and then 4.1 g of Pd/C was added. The temperature was controlled at 23° C. The system was replaced with nitrogen, and then hydrogen was introduced. After reacting for 23 hours, TLC showed that N-benzyloxycarbonyl-O-tert-butyl-L-seryl-O-tert-butyl-L-seryl-glycine benzyl ester had completely reacted. After filtering out the Pd/C, the filtrate was concentrated until a large amount of solid precipitated. The system was cooled to 2° C. for crystallization for 3 hours. The filter cake was rinsed with a small amount of methanol, and dried by blowing at 50° C. to dryness to obtain 20 g of O-tert-butyl-L-seryl-O-tert-butyl-L-seryl-glycine. The results show that: molar yield: 16%; HPLC: 99.28%; isomers: not detected.

### Comparative Example 1

This comparative Example uses N-benzyloxycarbonyl-O-tert-butyl-L-serine, O-tert-butyl-L-serine methyl ester and glycine methyl ester hydrochloride as main raw materials, and requires multiple synthesis steps. The obtained final compound O-tert-butyl-L-seryl-O-tert-butyl-L-seryl-glycine contains more impurities, is not easy to purify, and has a lower molar yield. This comparative Example has one more reaction step than the inventive examples.

### (1) Preparation of N-benzyloxycarbonyl-O-tert-butyl-L-seryl-O-tert-butyl-L-serine methyl ester

100 g of N-benzyloxycarbonyl-O-tert-butyl-L-serine was added into 800 g of ethyl acetate, and stirred to dissolve completely, and then 90 g of O-tert-butyl-L-serine methyl ester and 51.3 g of trimethylamine were added. After stirring uniformly, the mixture was cooled to 3° C., and then 9.1 g of HOBT and 78 g of EDCl were added. The mixture was stirred to react for 30 minutes, and heated to 33° C. to react for 18 hours. TLC showed that N-carbobenzoxy-O-tert-butyl-L-serine had completely reacted. 400 g of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 167 g of N-benzyloxycarbonyl-O-tert-butyl-L-seryl-O-tert-butyl-L-serine methyl ester as an oily substance.

### (2) Preparation of N-benzyloxycarbonyl-O-tert-butyl-L-seryl-O-tert-butyl-L-serine

167 g of the N-benzyloxycarbonyl-O-tert-butyl-L-seryl-O-tert-butyl-L-serine methyl ester as the oily substance was added into 150 g of tetrahydrofuran and 300 g of water. The mixture was cooled to 4° C., and 67.6 g of 30% sodium hydroxide aqueous solution was slowly added. After stirring uniformly, the mixture was heated to 23° C. to react for 3 hours. TLC showed that N-benzyloxycarbonyl-O-tert-butyl-L-seryl-O-tert-butyl-L-serine methyl ester had completely reacted. 400 g of ethyl acetate was added into the system. The pH value of the system was adjusted to 2-3 by using a 6N hydrochloric acid aqueous solution, and then liquids were separated. The ethyl acetate layer was collected. The ethyl acetate layer was washed with saturated sodium chloride for 3 times. After the washing was completed, the ethyl acetate layer was dried over sodium sulfate, filtered, and concentrated to dryness to obtain 153 g of the N-benzyloxycarbonyl-O-tert-butyl-L-seryl-O-tert-butyl-L-serine as an oily substance.

### (3) Preparation of N-benzyloxycarbonyl-O-tert-butyl-L-seryl-O-tert-butyl-L-seryl-glycine methyl ester

153 g of N-benzyloxycarbonyl-O-tert-butyl-L-seryl-O-tert-butyl-L-serine was added into 1200 g of ethyl acetate, and stirred to dissolve completely, and then 52.5 g of glycine methyl ester hydrochloride and 52.8 g of triethylamine were added. After stirring uniformly, the mixture was cooled to 2° C., and then 9.4 g of HOBT and 79.9 g of EDCl were added. The mixture was stirred to react for 30 minutes, and heated to 33° C. to react for 18 hours. TLC showed that N-benzyloxycarbonyl-O-tert-butyl-L-seryl-O-tert-butyl-L-serine had completely reacted. 400 g of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 157 g of N-benzyloxycarbonyl-O-tert-butyl-L-seryl-O-tert-butyl-L-seryl-glycine methyl ester as an oily substance.

### (4) Preparation of N-benzyloxycarbonyl-O-tert-butyl-L-seryl-O-tert-butyl-L-seryl-glycine

157 g of the N-benzyloxycarbonyl-O-tert-butyl-L-seryl-O-tert-butyl-L-seryl-glycine methyl ester as the oily substance was added into 160 g of tetrahydrofuran and 300 g of water. The mixture was cooled to 4° C., and 61.7 g of 30% sodium hydroxide aqueous solution was slowly added. After stirring uniformly, the mixture was heated to 23° C. to react for 3 hours. TLC showed that N-benzyloxycarbonyl-O-tert-butyl-L-seryl-O-tert-butyl-L-seryl-glycine methyl ester had completely reacted. 400 g of ethyl acetate was added into the system. The pH value of the system was adjusted to 2-3 by using a 6N hydrochloric acid aqueous solution, and then liquids were separated. The ethyl acetate layer was collected. The ethyl acetate layer was washed with saturated sodium chloride for 3 times. After the washing was completed, the ethyl acetate layer was dried over sodium sulfate, filtered, and concentrated to dryness to obtain 138 g of N-benzyloxycarbonyl-O-tert-butyl-L-seryl-O-tert-butyl-L-seryl-glycine as an oily substance.

### (5) Preparation of O-tert-butyl-L-seryl-O-tert-butyl-L-seryl-glycine

138 g of N-benzyloxycarbonyl-O-tert-butyl-L-seryl-O-tert-butyl-L-seryl-glycine was added to 828 g of methanol, and stirred to dissolve completely, and then 13.8 g of Pd/C was added. The temperature was controlled at 23° C. The system was replaced with nitrogen, and then hydrogen was introduced. After reacting for 23 hours, TLC showed that N-benzyloxycarbonyl-O-tert-butyl-L-seryl-O-tert-butyl-L-seryl-glycine had completely reacted. After filtering out the Pd/C, the filtrate was concentrated until a large amount of solid precipitated. The system was cooled to 2° C. for crystallization for 3 hours. The filter cake was rinsed with a small amount of methanol, and dried by blowing at 50° C. to dryness to obtain 45 g of O-tert-butyl-L-seryl-O-tert-butyl-L-seryl-glycine. The results show that: molar yield: 37%; HPLC: 93.6%; isomers: not detected.

### Comparative Example 2

This comparative Example uses N-tert-butoxycarbonyl-O-tert-butyl-L-serine, O-tert-butyl-L-serine methyl ester and glycine methyl ester hydrochloride as main raw materials, and requires multiple synthesis steps. The obtained final compound O-tert-butyl-L-seryl-O-tert-butyl-L-seryl-glycine contains more impurities, is not easy to purify, and has a lower molar yield. This comparative Example has one more reaction step than the inventive examples.

### (1) Preparation of N-tert-butoxycarbonyl-O-tert-butyl-L-seryl-O-tert-butyl-L-serine methyl ester

100 g of N-tert-butoxycarbonyl-O-tert-butyl-L-serine was added into 800 g of ethyl acetate, and stirred to dissolve completely, and then 100 g of O-tert-butyl-L-serine methyl ester and 54.2 g of trimethylamine were added. After stirring uniformly, the mixture was cooled to 3° C., and then 7.5 g of HOBT and 88 g of EDCl were added. The mixture was stirred to react for 30 minutes, and heated to 33° C. to react for 18 hours. TLC showed that N-carbobenzoxy-O-tert-butyl-L-serine had completely reacted. 400 g of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 157 g of N-tert-butoxycarbonyl-O-tert-butyl-L-seryl-O-tert-butyl-L-serine methyl ester as an oily substance.

### (2) Preparation of N-tert-butoxycarbonyl-O-tert-butyl-L-seryl-O-tert-butyl-L-serine

157 g of the N-tert-butoxycarbonyl-O-tert-butyl-L-seryl-O-tert-butyl-L-serine methyl ester as the oily substance was added into 150 g of tetrahydrofuran and 300 g of water. The mixture was cooled to 4° C., and 75 g of 30% sodium hydroxide aqueous solution was slowly added. After stirring uniformly, the mixture was heated to 23° C. to react for 3 hours. TLC showed that N-tert-butoxycarbonyl-O-tert-butyl-L-seryl-O-tert-butyl-L-serine methyl ester had completely reacted. 400 g of ethyl acetate was added into the system. The pH value of the system was adjusted to 2-3 by using a 6N hydrochloric acid aqueous solution, and then liquids were separated. The ethyl acetate layer was collected. The ethyl acetate layer was washed with saturated sodium chloride for 3 times. After the washing was completed, the ethyl acetate layer was dried over sodium sulfate, filtered, and concentrated to dryness to obtain 136 g of N-tert-butoxycarbonyl-O-tert-butyl-L-seryl-O-tert-butyl-L-serine as an oily substance.

### (3) Preparation of N-tert-butoxycarbonyl-O-tert-butyl-L-seryl-O-tert-butyl-L-seryl-glycine methyl ester

136 g of N-tert-butoxycarbonyl-O-tert-butyl-L-seryl-O-tert-butyl-L-serine was added into 1100 g of ethyl acetate, and stirred to dissolve completely, and then 50.7 g of glycine methyl ester hydrochloride and 47.6 g of triethylamine were added. After stirring uniformly, the mixture was cooled to 2° C., and then 9 g of HOBT and 77 g of EDCl were added. The mixture was stirred to react for 30 minutes, and heated to 33° C. to react for 18 hours. TLC showed that N-tert-butoxycarbonyl-O-tert-butyl-L-seryl-O-tert-butyl-L-serine had completely reacted. 400 g of water was added to the reaction solution to quench the reaction, and then liquids were separated. Ethyl acetate layer was collected. The ethyl acetate layer was washed with 6N hydrochloric acid aqueous solution, 5% sodium bicarbonate aqueous solution and saturated sodium chloride aqueous solution respectively. After the washing was completed, the ethyl acetate layer was dried over sodium sulfate, filtered, and concentrated to dryness to obtain 148 g of N-tert-butoxycarbonyl-O-tert-butyl-L-seryl-O-tert-butyl-L-seryl-glycine methyl ester as an oily substance.

### (4) Preparation of N-tert-butoxycarbonyl-O-tert-butyl-L-seryl-O-tert-butyl-L-seryl-glycine

148 g of the N-tert-butoxycarbonyl-O-tert-butyl-L-seryl-O-tert-butyl-L-seryl-glycine methyl ester as the oily substance was added into 150 g of tetrahydrofuran and 300 g of water. The mixture was cooled to 4° C., and 62.3 g of 30% sodium hydroxide aqueous solution was slowly added. After stirring uniformly, the mixture was heated to 23° C. to react for 3 hours. TLC showed that N-tert-butoxycarbonyl-O-tert-butyl-L-seryl-O-tert-butyl-L-seryl-glycine methyl ester had completely reacted. 400 g of ethyl acetate was added into the system. The pH value of the system was adjusted to 2-3 by using a 6N hydrochloric acid aqueous solution, and then liquids were separated. The ethyl acetate layer was collected. The ethyl acetate layer was washed with saturated sodium chloride for 3 times. After the washing was completed, the ethyl acetate layer was dried over sodium sulfate, filtered, and concentrated to dryness to obtain 132 g of N-tert-butoxycarbonyl-O-tert-butyl-L-seryl-O-tert-butyl-L-seryl-glycine as an oily substance.

### (5) Preparation of O-tert-butyl-L-seryl-O-tert-butyl-L-seryl-glycine

132 g of N-tert-butoxycarbonyl-O-tert-butyl-L-seryl-O-tert-butyl-L-seryl-glycine was added to 660 g of dichloromethane, and stirred to dissolve completely, and then 65.3 g of trifluoroacetic acid was added. The temperature was controlled at 23° C. After reacting for 4 hours, TLC showed that N-tert-butoxycarbonyl-O-tert-butyl-L-seryl-O-tert-butyl-L-seryl-glycine had completely reacted. The dichloromethane and trifluoroacetic acid were concentrated to dryness. Then 500 g of methanol was added. The system was cooled to 7° C. The pH value of the system was adjusted to 6-7 by using 35 g of triethylamine. Then the system was cooled to 2° C. for crystallization for 3 hours, and then filtered. The filter cake was rinsed with a small amount of methanol, and dried by blowing at 50° C. to dryness to obtain 34 g of O-tert-butyl-L-seryl-O-tert-butyl-L-seryl-glycine. The results show that: molar yield: 28%; HPLC: 95.3%; isomers: not detected.

Table 1 provides data on the yield and purity under different raw material selections for Example 1, Comparative Example 1, and Comparative Example 2.

TABLE 1
Data on Effect of Different Main Raw Materials on the Yield and Purity of the Target Product

| | Main Raw Material 1 | Main Raw Material 2 | Main Raw Material 3 | Yield | Purity |
|---|---|---|---|---|---|
| Example 1 | N-benzyloxycarbonyl- | O-tert-butyl-L-serine | Glycine benzyl | 73% | 99.48% |
| | O-tert-butyl-L-serine | methyl ester | ester | | |
| | | | p-toluenesulfonate | | |
| Comparative | N-benzyloxycarbonyl- | O-tert-butyl-L-serine | Glycine methyl | 37% | 93.6% |
| Example 1 | O-tert-butyl-L-serine | methyl ester | ester hydrochloride | | |
| Comparative | N-tert-butoxycarbonyl- | O-tert-butyl-L-serine | Glycine methyl | 28% | 95.3% |
| Example 2 | O-tert-butyl-L-serine | methyl ester | ester hydrochloride | | |

### Example 5

Example 5 investigates the effect of condensing agents on the target product. Specifically, the preparation method is the same as that of Example 1, except that the types of the condensing agents in step (1) and step (3) are changed. Through experiments, the yield and purity of the obtained target product are shown in Table 2 below.

TABLE 2
Data on Yield and Purity of the Target Product
Under Different Condensing Agent Selections

| | Condensing Agent | Yield | Purity |
|---|---|---|---|
| Experimental Group 1 | EDCl/HOBT | 73% | 99.48% |
| (Example 1) | | | |
| Experimental Group 2 | EDCl/HOSU | 68% | 99.37% |
| Experimental Group 3 | DCC/HOSU | 54% | 97.68% |
| Experimental Group 4 | HATU | 69% | 99.33% |
| Experimental Group 5 | HBTU | 68% | 99.36% |
| Experimental Group 6 | CDI | 38% | 98.42% |

### Example 6

Example 6 investigates the effect of temperature of condensation reaction on the target product. Specifically, the preparation method is the same as that of Example 1, except that the temperature of the condensation reaction is changed. Through experiments, the yield and purity of the obtained target product are shown in Table 3 below.

TABLE 3
Data on Yield and Purity of the Target Product
at Different Condensation Temperatures

| | Temperature of | | |
|---|---|---|---|
| | Condensation | | |
| | Reaction/° C. | Yield | Purity |
| Experimental Group 7 | 0-10 | 16% | 99.28% |
| (Example 4) | | | |
| Experimental Group 8 | 10-20 | 32% | 99.34% |
| Experimental Group 9 | 20-25 | 54% | 99.44% |
| Experimental Group 10 | 25-30 | 64% | 99.41% |
| Experimental Group 1 | 30-35 | 73% | 99.48% |
| (Example 1) | | | |
| Experimental Group 11 | 35-40 | 75% | 98.42% |
| Experimental Group 12 | 40-50 | 74% | 96.26% |

### Example 7

Example 7 investigates the effect of organic solvents on the target product. Specifically, the preparation method is the same as that of Example 1, except that the selection of organic solvents in step (1) and step (3) is changed. Through experiments, the yield and purity of the obtained target product are shown in Table 4 below.

TABLE 4
Data on Yield and Purity of the Target
Product with Different Organic Solvents

| | Organic Solvent | Yield | Purity |
|---|---|---|---|
| Experimental Group 1 | Ethyl Acetate | 73% | 99.48% |
| (Example 1) | | | |
| Experimental Group 13 | Tetrahydrofuran | 55% | 99.14% |
| Experimental Group 14 | Dichloromethane | 46% | 98.44% |
| Experimental Group 15 | Acetonitrile | 62% | 99.06% |

### Example 8

Example 8 investigates the effect of concentration of sodium hydroxide on the target product. Specifically, the preparation method is the same as that of Example 1, except that the concentration of sodium hydroxide solution in step (2) is changed. Through experiments, the reaction time (the reaction time of step (2)) and the purity of the obtained target product are shown in Table 5 below.

TABLE 5
Data on Reaction Time and Product Purity at Different
Concentrations of Sodium Hydroxide Solution

| | Concentration of | Reaction | |
|---|---|---|---|
| | Sodium Hydroxide | Time | Purity |
| Experimental Group 16 | 10% Sodium Hydroxide | 7 h | 99.38% |
| Experimental Group 17 | 20% Sodium Hydroxide | 5 h | 99.42% |
| Experimental Group 1 | 30% Sodium Hydroxide | 3 h | 99.48% |
| (Example 1) | | | |
| Experimental Group 18 | 40% Sodium Hydroxide | 2 h | 98.11% |
| Experimental Group 19 | 50% Sodium Hydroxide | 1 h | 95.54% |
| Experimental Group 20 | 60% Sodium Hydroxide | 0.5 h | 92.16% |

### Example 9

Example 9 investigates the effect of temperature of saponification reaction on the target product. Specifically, the preparation method is the same as that of Example 1, except that the temperature of the saponification reaction in step (2) is changed. Through experiments, the reaction time (the reaction time of step (2)) and the purity of the obtained target product are shown in Table 6 below.

TABLE 6
Data on Reaction Time and Product Purity at Different
Temperatures of the Saponification Reaction

| | Temperature of | | |
|---|---|---|---|
| | Saponification | | |
| | Reaction/° C. | Reaction Time | Purity |
| Experimental Group 21 | 0-10 | 6 h | 99.48% |
| Experimental Group 22 | 10-15 | 5 h | 99.46% |
| Experimental Group 23 | 15-20 | 4 h | 99.49% |
| Experimental Group 1 | 20-25 | 3 h | 99.48% |
| (Example 1) | | | |
| Experimental Group 24 | 25-30 | 2 h | 98.37% |
| Experimental Group 25 | 30-40 | 1 h | 96.74% |

In summary, the present application provides a method for preparing the tirzepatide intermediate O-tert-butyl-L-seryl-O-tert-butyl-L-seryl-glycine, which is suitable for scaled-up production. The synthesis method requires a simple equipment, has a good reaction efficiency, and a low production cost, and the final product has a purity of higher than 99.0%.

The above description of the examples is only used to help understand the method of the present application and its core idea. It should be pointed out that several improvements and modifications can be made to the present application without departing from the principles of the present application, and these improvements and modifications also fall within the protection scope of the claims of the present application.

Various modifications to these examples may be made in alignment with the general principles defined herein can be implemented in other examples without departing from the spirit or scope of the present application. Therefore, the present application will not be limited to the examples shown herein, but is to conform to the widest scope consistent with the principles and novel features disclosed herein.

1. O-tert-butyl-L-seryl-O-tert-butyl-L-seryl-glycine represented by formula (I)

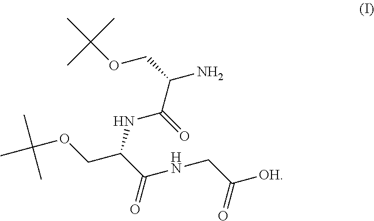 [SMILES: CC(C)(C)OC[C@H](NC(=O)[C@@H](N)COC(C)(C)C)C(=O)NCC(=O)O]

2. A method for preparing the O-tert-butyl-L-seryl-O-tert-butyl-L-seryl-glycine according to claim 1, comprising the following steps:
S1) carrying out a condensation reaction on N-benzyloxycarbonyl-O-tert-butyl-L-serine and O-tert-butyl-L-serine methyl ester under the action of a condensing agent and an organic base to obtain N-benzyloxycarbonyl-O-tert-butyl-L-seryl-O-tert-butyl-L-serine methyl ester represented by formula (II);
S2) carrying out a saponification reaction on the N-benzyloxycarbonyl-O-tert-butyl-L-seryl-O-tert-butyl-L-serine methyl ester and an alkaline compound to obtain N-benzyloxycarbonyl-O-tert-butyl-L-seryl-O-tert-butyl-L-serine represented by formula (III);
S3) carrying out a condensation reaction on the N-benzyloxycarbonyl-O-tert-butyl-L-seryl-O-tert-butyl-L-serine and glycine benzyl ester p-toluenesulfonate under the action of a condensing agent and an organic base to obtain N-benzyloxycarbonyl-O-tert-butyl-L-seryl-O-tert-butyl-L-seryl-glycine benzyl ester represented by formula (IV);
S4) carrying out a hydrogenation reaction on the N-benzyloxycarbonyl-O-tert-butyl-L-seryl-O-tert-butyl-L-seryl-glycine benzyl ester and hydrogen under the action of a catalyst to obtain O-tert-butyl-L-seryl-O-tert-butyl-L-seryl-glycine represented by formula (I);

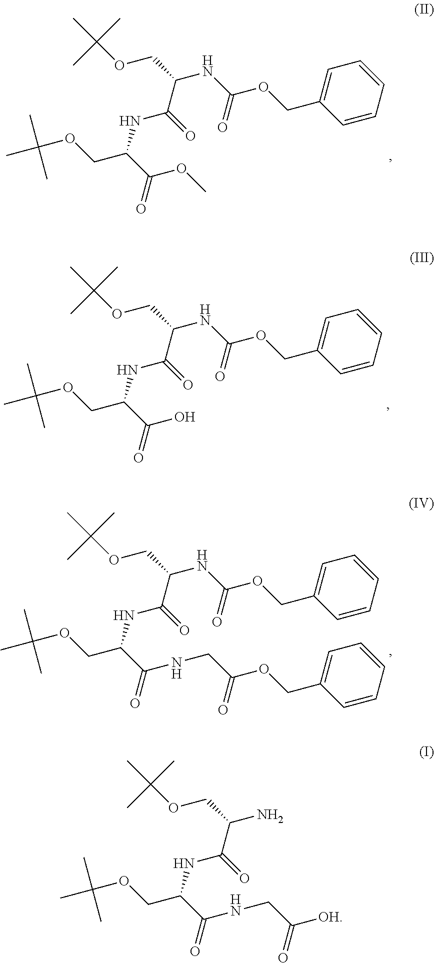 [SMILES: COC(=O)[C@H](COC(C)(C)C)NC(=O)[C@H](COC(C)(C)C)NC(=O)OCc1ccccc1 ; CC(C)(C)OC[C@H](NC(=O)[C@H](COC(C)(C)C)NC(=O)OCc1ccccc1)C(=O)O ; CC(C)(C)OC[C@H](NC(=O)OCc1ccccc1)C(=O)N[C@@H](COC(C)(C)C)C(=O)NCC(=O)OCc1ccccc1 ; CC(C)(C)OC[C@H](NC(=O)[C@@H](N)COC(C)(C)C)C(=O)NCC(=O)O]

3. The method according to claim 2, wherein step S1) specifically is:
mixing the N-benzyloxycarbonyl-O-tert-butyl-L-serine, the O-tert-butyl-L-serine methyl ester and the organic base with an organic solvent, then adding the condensing agent to carry out the condensation reaction to obtain the N-benzyloxycarbonyl-O-tert-butyl-L-seryl-O-tert-butyl-L-serine methyl ester.

4. The method according to claim 2, wherein in step S1), a molar ratio of the N-benzyloxycarbonyl-O-tert-butyl-L-serine, the O-tert-butyl-L-serine methyl ester, the condensing agent, and the organic base is 1:(1-2):(1-1.5):(1-1.5).

5. The method according to claim 2, wherein the condensing agent is selected from the group consisting of EDCl/HOBT, EDCl/HOSU, DCC/HOSU, HATU, HBTU and CDI, and the organic base is selected from the group consisting of triethylamine and N,N-diisopropylethylamine.

6. The method according to claim 3, wherein the condensing agent is added at a temperature of 0-5° C., and the condensation reaction is carried out at a temperature of 0-50° C. for 12-24 h.

7. The method according to claim 3, wherein the organic solvent is selected from the group consisting of ethyl acetate, dichloromethane, acetonitrile and tetrahydrofuran.

8. The method according to claim 2, wherein step S2) specifically is:
mixing the N-benzyloxycarbonyl-O-tert-butyl-L-seryl-O-tert-butyl-L-serine methyl ester with an organic solvent, then adding a sodium hydroxide solution to carry out the saponification reaction to obtain the N-benzyloxycarbonyl-O-tert-butyl-L-seryl-O-tert-butyl-L-serine.

9. The method according to claim 8, wherein in step S2), a molar ratio of the N-benzyloxycarbonyl-O-tert-butyl-L-seryl-O-tert-butyl-L-serine methyl ester to the sodium hydroxide is 1:(1-2).

10. The method according to claim 8, wherein in step S2), the sodium hydroxide solution is added at a temperature of 0-5° C., and the saponification reaction is carried out at a temperature of 0-40° C. for 1-6 h.

11. The method according to claim 8, wherein in step S2), a concentration of the sodium hydroxide solution is 5-60%.

12. The method according to claim 8, wherein in step S2), the organic solvent is selected from the group consisting of acetonitrile, tetrahydrofuran, methanol and ethanol.

13. The method according to claim 2, wherein step S3) specifically is:
mixing the N-benzyloxycarbonyl-O-tert-butyl-L-seryl-O-tert-butyl-L-serine, the glycine benzyl ester p-toluenesulfonate and the organic base with an organic solvent, then adding the condensing agent to carry out the condensation reaction to obtain the N-benzyloxycarbonyl-O-tert-butyl-L-seryl-O-tert-butyl-L-seryl-glycine benzyl ester.

14. The method according to claim 2, wherein in step S3), a molar ratio of the N-benzyloxycarbonyl-O-tert-butyl-L-seryl-O-tert-butyl-L-serine, the glycine benzyl ester p-toluenesulfonate, the organic base, and the condensing agent is 1:(1-2):(1-1.5):(1-1.5).

15. The method according to claim 2, wherein in step S3), the condensing agent is selected from the group consisting of EDCl/HOBT, EDCl/HOSU, DCC/HOSU, HATU, HBTU and CDI, and the organic base is selected from the group consisting of trimethylamine and N, N-diisopropylethylamine.

16. The method according to claim 13, wherein in step S3), the condensing agent is added at a temperature of 0-5° C., and the condensation reaction is carried out at a temperature of 0-50° C. for 12-24 h.

17. The method according to claim 13, wherein in step S3), the organic solvent is selected from the group consisting of ethyl acetate, dichloromethane, acetonitrile and tetrahydrofuran.

18. The method according to claim 2, wherein step S4) specifically is:
mixing the N-benzyloxycarbonyl-O-tert-butyl-L-seryl-O-tert-butyl-L-seryl-glycine benzyl ester with an organic solvent, adding the catalyst, and then introducing hydrogen to carry out the hydrogenation reaction to obtain the O-tert-butyl-L-seryl-O-tert-butyl-L-seryl-glycine.

19. The method according to claim 2, wherein in step S4), a mass ratio of the N-benzyloxycarbonyl-O-tert-butyl-L-seryl-O-tert-butyl-L-seryl-glycine benzyl ester to the catalyst is 1:(0.01-0.2); or
wherein in step S4), the hydrogenation reaction is carried out at a temperature of 20-30° C. for 20-24 h.

20. The method according to claim 18, wherein the organic solvent is selected from the group consisting of acetonitrile, tetrahydrofuran/water, methanol, ethanol, acetone/water and ethyl acetate/water, and the catalyst is selected from the group consisting of Pd/C, Raney nickel and Pd/BaSO₄.
